(19) United States
(12) Patent Application Publication
(10) Pub. No.: US 2022/0246281 A1
(43) Pub. Date: Aug. 4, 2022

(54) METHODS AND SYSTEMS FOR IMPROVING RADIOLOGY WORKFLOW

(71) Applicant: AVICENNA.AI, Marseille (FR)

(72) Inventor: Cyril DI GRANDI, La Ciotat (FR)

(73) Assignee: AVICENNA.AI, Marseille (FR)

(21) Appl. No.: 17/162,306

(22) Filed: Jan. 29, 2021

(51) Int. Cl. G16H 30/20; G06F 16/58; G06F 16/54; G06F 16/538; G06T 7/00

(52) U.S. Cl. CPC G16H 30/20; G06F 16/5866; G06T 7/0012; G06F 16/538; G06F 16/54

(57) ABSTRACT

A system to enable application of a function or service to a medical image when displayed by a medical image viewer, including: a tagging unit configured to apply a visual tag on the medical image so that the visual tag is visible when a tagged medical image is displayed by the medical image viewer, the visual tag containing information relating to the function or service; and an application unit configured to identify the applied visual tag when the tagged medical image is displayed by the medical image viewer, the application unit being further able to apply the function or service to the medical image based on information contained in the identified visual tag.

### FIELD OF THE INVENTION

The present invention pertains generally to medical diagnostic imaging systems and more particularly to medical image viewers such as Digital Imaging and Communications in Medicine (DICOM) viewers and the like.

### BACKGROUND OF THE INVENTION

DICOM is a leading standard for digital image data management in medical applications. It is used to capture, exchange, and archive image data in Picture Archiving and Communication Systems (PACS).

DICOM viewers which provide users (radiologist, physician and operator) with visualization tools of medical images are intended to support rapid and efficient diagnostic imaging workflow. They assume a central role in radiology workflow as they are a decisive factor for diagnostic quality and drawn conclusions.

In this regard, physicians use dedicated workstations connected to the PACS and equipped with specialized medical image viewers to retrieve and visualize medical images produced by various modalities such as computed radiography (CR), magnetic resonance images (MRI), computed tomography (CT), or ultrasound (U/S). Any user interaction with such medical images should be achieved through the graphical user interfaces of the DICOM viewer in use.

Whether they are standalone software or application components, DICOM viewers with advanced functionalities and visualization solutions are constantly emerging to assist medical images interpretation and reporting in different medical specialties. Accordingly, there is a continuing need to integrate new functionalities and/or services (e.g., post processing, segmentation, annotation, image reconstruction, calculation, viewing, etc.) driven by diagnostic needs, work processes simplification and/or software technologies evolution. To that end, one solution is to update installed DICOM viewer software whenever necessary.

However, many challenging problems may be encountered in attempting to modify DICOM viewer software which has been in use for a long time.

In fact, to be able to modify DICOM viewer software, the source code and appropriate documents and support should be available. In addition, in a same medical environment, diverse platform-dependent and/or manufacturer-specific DICOM viewer software may exist with elaborate setup resulting in each software component having to be independently dealt with. Even if it is possible, this is obviously time-consuming and not profitable to the hospital.

Furthermore, continuous modification of software may lead to an accumulation of not-natively supported extensions (not foreseen beforehand) which may both degrade the DICOM viewer reliability and performance as well as reduce the effectiveness of radiologists.

Another problem arises with regard to medical applications where it is quite essential to take into consideration user-friendliness, usability and familiarity with graphic user interfaces. Modifying the viewer appearance or adding new graphic user interfaces may distort already acquired interaction practice of the physician with the DICOM viewer and consequently impair radiology workflow and reports relevance. The subjective assessment, experience or computer skills of physicians may impede such modification of viewer tools.

As a further problem, physicians are usually used to viewing mainly two graphical user interfaces; a worklist and a medical image. Therefore, any additional user interface for diagnostic purposes could be considered cumbersome and inconvenient.

### SUMMARY

Various embodiments are directed to addressing the effects of one or more of the problems set forth above. The following presents a simplified summary of embodiments in order to provide a basic understanding of some aspects of the various embodiments. This summary is not an exhaustive overview of these various embodiments. It is not intended to identify key or critical elements or to delineate the scope of these various embodiments. Its sole purpose is to present some concepts in a simplified form as a prelude to the more detailed description that is discussed later.

Some embodiments overcome one or more drawbacks of the prior art, by providing user-friendly and intuitive systems for implementing front-end functionalities on medical image viewers.

Some embodiments provide efficient, simple, and practical amendment methods of medical image viewer software while preserving user experience as much as possible.

Some embodiments provide DICOM viewers software including advanced functionalities to meet the needs of physicians in specific use case scenarios.

Various embodiments relate to a system to enable application of a feature to a medical image when displayed by a medical image viewer, said system including:
a tagging unit configured to apply a visual tag on said medical image so that the visual tag is visible when the tagged medical image is displayed by the medical image viewer, said visual tag comprising information relating to said feature; and
an application unit able to identify the applied visual tag when the tagged medical image is displayed by the medical image viewer, said application unit being further able to apply said feature to the medical image based on information contained in the identified visual tag.

In accordance with a broad aspect, the application unit comprises a mobile user equipment including:
an image sensor for capturing an image of at least a portion of the displayed tagged medical image, said at least a portion comprising the visual tag; and
a mobile application for identifying the visual tag contained in the captured image, and for determining from the identified visual tag information relating to said feature, the mobile application being able to apply said feature.

In accordance with another broad aspect, the system further includes a user terminal, the user terminal comprising the medical image viewer, the user terminal further comprising the application unit.

In accordance with another broad aspect, the application unit comprises a software application configured to detect and identify the visual tag when the tagged medical image is displayed by the medical image viewer, said software application being further configured to detect a predefined user interaction with the identified visual tag, said software application being able to apply said feature when the predefined user interaction is detected.

In accordance with another broad aspect, the medical image viewer is supported by a web browser, the software application being a plug-in associated with the web browser.

In accordance with another broad aspect, the medical image is a Digital Imaging and Communications in Medicine object.

In accordance with another broad aspect, the information relating to the feature comprises a query/retrieve request.

In accordance with another broad aspect, the medical image comprises a top layer, the visual tag being applied to the top layer.

In accordance with another broad aspect, various embodiments relate to a method to enable application of a feature to a medical image when displayed by a medical image viewer, said method including the following steps:
applying a visual tag to said medical image so that the visual tag is visible when tagged medical image is displayed by the medical image viewer, said visual tag comprising information relating to said feature;
identification of the applied visual tag when the tagged medical image is displayed by the medical image viewer, and
applying said feature to the medical image based on information contained in the identified visual tag.

In accordance with another broad aspect, the method further comprises the following steps:
capturing an image of at least a portion of the displayed tagged medical image, said at least a portion comprising the visual tag;
identification of the visual tag contained in the captured image; and
determination from the identified visual tag information relating to said feature.

In accordance with another broad aspect, the method further comprises the following steps:
detection and identification of the visual tag when the tagged medical image is displayed by the medical image viewer; and
detection of a predefined user interaction with the identified visual tag, said feature being applied to the medical image when the predefined user interaction is detected.

In accordance with another broad aspect, various embodiments relate to a medical imaging network comprising a medical imaging equipment, a medical image database system, a medical image viewer, the presented above system.

In accordance with another broad aspect, the medical imaging network is a Digital Imaging and Communications in Medicine network.

While the various embodiments are susceptible to various modification and alternative forms, specific embodiments thereof have been shown by way of example in the drawings. It should be understood, however, that the description herein of specific embodiments is not intended to limit the various embodiments to the particular forms disclosed.

It may of course be appreciated that in the development of any such actual embodiments, implementation-specific decisions should be made to achieve the developer's specific goal, such as compliance with system-related and business-related constraints. It will be appreciated that such a development effort might be time consuming but may nevertheless be a routine understanding for those or ordinary skill in the art having the benefit of this disclosure.

### DESCRIPTION OF THE DRAWING

The objects, advantages and other features of the present invention will become more apparent from the following disclosure and claims. The following non-restrictive description of preferred embodiments is given for the purpose of exemplification only with reference to the accompanying drawing in which

FIG. 1 schematically illustrates elements of a medical imaging network according to various embodiments;

FIG. 2 schematically illustrates process steps of a method to enable application of a feature to a medical image when displayed by a medical image viewer according to various embodiments;

FIG. 3 schematically illustrates elements of a computing device according to various embodiments.

### DETAILED DESCRIPTION OF ILLUSTRATIVE EMBODIMENTS

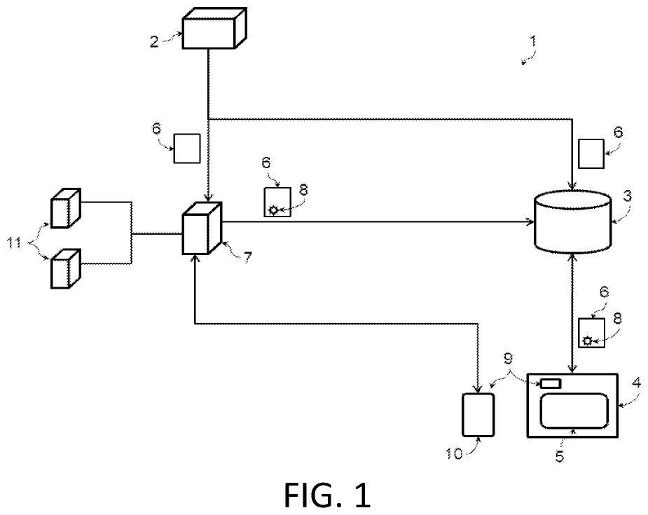

With reference to FIG. 1, there is shown a medical imaging network 1 comprising a medical imaging equipment 2, a medical image database system 3 (or a medical image storage system), and at least a user terminal 4 including a medical image viewer 5.

The medical imaging network 1 is configured to enable the handling of medical images from their acquisition by means of the medical imaging equipment 2 to their visualization by means of the medical image viewer 5. The medical imaging network 1 is, for instance, the intranet of a local Area Network (LAN) of a hospital's radiology IT network. The medical imaging network 1 can be devoid of external access or provide access to stored medical images via, for example, Web protocols.

The medical imaging network 1 uses a predefined protocol for acquiring, storing, transmitting, retrieving and displaying medical imaging data. In one embodiment, this protocol is DICOM. Accordingly, the medical imaging network 1 is a DICOM network where medical images 6 are captured, stored, transmitted, and displayed as DICOM objects.

The medical imaging equipment 2 is any diagnostic system for medical image 6 record, capture, generation, or acquisition. The medical imaging equipment 2 may be of any modality such as Digital Radiography (DX), Magnetic Resonance (MR), Ultrasound (US), Endoscopy (ES), Laser surface scan (LS), Positron emission Tomography (PT), Mammography (MG), X-Ray Angiography (XA) or the like. The medical imaging equipment 2 may be a device that includes one or more sensors to capture an image of a physical object (e.g., a bodily object) using one or more of the above-listed modalities.

In addition to providing the image, the medical imaging equipment 2 may generate or acquire metadata including, for example, patient information (such as a name, an identifier, a gender, or a date of birth), data about the image (such as the imaged body part, an annotation, the date and time of capture, the image dimensions or resolution) and information about the settings of the medical imaging equipment 2 used for obtaining the medical image 6. The metadata and corresponding image are stored in a single file. For instance, a DICOM image includes text-based metadata called a DICOM header.

The medical image database system 3 is configured to store digital medical images and related information. The medical image database system 3 allows querying and retrieving medical images 6 and corresponding metadata stored therein. The medical image database system 3 may be a centralized repository or a plurality of decentralized repositories configured to store, for instance, medical images 6 produced in a radiology department or, more generally, in a hospital environment. In one embodiment, the medical image database system 3 is a PACS.

The user terminal 4 is a radiologist workstation, a radiologist console, a dedicated or multipurpose computer, or more generally any fixed or mobile device including a medical image viewer 5 installed thereon and connected in a wireless and/or wired manner to the medical imaging network 1.

The medical image viewer 5 includes a graphical user interface (GUI) for medical images visualization and possibly annotation. In one embodiment, the medical image viewer 5 is a standalone software (e.g., a desktop application). The medical image viewer 5 may be a desktop based viewer such as a PC-based software or a web-based viewer (i.e. supported by a web browser or a web client). In one embodiment, the medical image viewer 5 is a DICOM viewer for the display of DICOM image files on a computer monitor.

The medical image viewer 5 includes tools for viewing and manipulating medical images 6 including zooming, rotating, or taking measurements. The medical image viewer 5 may further include annotation tools like freehand Region of Interest (RoI) marking.

The medical image viewer 5 enables physicians to retrieve, visualize, explore and annotate a medical image 6. In one embodiment, the medical imaging equipment 2 is equipped with a medical image viewer 5.

Regarding data flow, medical images 6 are sent from the medical imaging equipment 2 to the medical image database system 3 where medical images 6 can be queried by the medical image viewer 5 installed on the user terminal 4.

The medical imaging network 1 further includes a tagging unit 7 configured to apply a visual tag 8 (or a graphic tag) to an incoming medical image 6. The visual tag 8 is applied to the medical image 6 so that it is visible when the tagged medical image is displayed by the medical image viewer 5. In other words, the visual tag 8 is applied to the medical image 6 so that it can be visualized by the physician when the tagged medical image 6 is displayed by the medical image viewer 5. The visual tag 8 is therefore directly shown to the physician.

The tagging unit 7 is a server side gateway or middleware configured to apply, affix, add, or append a visual tag 8 to a medical image 6. The visual tag 8 may comprise a text and/or a graphic. In one embodiment, the visual tag 8 is a QR-code, a Maxicode, a cybercode, a barcode, a pattern, a label, a symbol, an icon, a pictogram, a design, a text or any combination thereof.

In one embodiment, the tagging unit 7 supports DICOM services so that it can handle DICOM objects and communicate with any node of a DICOM network, particularly with a DICOM medical imaging equipment, a PACS, or a DICOM viewer.

Accordingly, the tagging unit 7 is able to apply (or append) a visual tag 8 to an incoming medical image 6 obtained by the medical imaging equipment 2 and uploads or transmits the tagged medical image 6 to the medical image database system 3. In the embodiment illustrated by FIG. 1, the medical image database system 3 comprises, for each tagged medical image 6, the original non-tagged medical image 6 (i.e., a clean or non-tagged copy) which is directly received from the medical imaging equipment 2. That is to say, a first and a second copy (or a copy and the original) of an obtained medical image 6 by the medical imaging equipment 2 are, respectively, transmitted to the tagging unit 7 and to the medical image database system 3.

In one embodiment, the tagging unit 7 applies the visual tag to a top layer (a transparent upper layer) of the medical image 6. The visual tag 8 is shown as a text and/or graphic overlay on the medical image 6. In one embodiment, the top layer including the visual tag 8 may be hidden (deactivated) or shown according to user preference settings in the medical image viewer 5. The medical image 6 includes multiple layers for viewing that are superimposed on one another. In some instances, the bottom layer may include image data of the medical image 6. In some instances, rather than applying the visual tag to the top layer, the tag may be applied to another layer superimposed with the image data. The layer may be transparent or not and/or include clear windows. Each of the upper layers (e.g., overlays) may be masked or not, independently. Each layer may be independently selectable for viewing, and annotated with comments relating to the patient, another dedicated modality acquisition, etc.

Different areas of the medical image 6 may have different priority levels. The visual tag 8 overlays an area of the medical image 6 which has the lowest priority during diagnosis. This area may be defined in a fixed way according to the dimensions of medical image 6 or determined by the tagging unit 7 according to the content of the medical image 6. As one example, the position of the visual tag 8 in the medical image 6 may be preset using pixel coordinates. For instance, the visual tag 8 can be placed at or within a predetermined distance (e.g., 1 centimeter) of a predefined edge or corner of the medical image 6 (away from the imaged body part, which has a higher priority). In another embodiment, a lossless area of the medical image 6 on which the visual tag 8 may be placed is determined by calculating the difference between the original and visually tagged medical image 6. The lossless area may be a spare area or portion of the medical image 6 that does not include any image data, or least not image data of the patient. More generally, the medical image 6 comprises a layer to which is appended or applied the visual tag 8. In some instances, the visual tag 8 may be adjusted in size to fit within the lossless area. In some instances, the visual tag 8 may be placed in a non-lossless area (e.g., the middle) of the medical image 6. In such instances, the visual tag 8 may be overlaid in its own layer that can be hidden or viewed as desired.

In one embodiment, the area or portion of the medical image 6 containing the visual tag 8 is determined according to information retrieved from the metadata of the medical image 6, such as the dimensions, the modality, or the imaged body part. To improve its visibility, the colors of the visual tag 8 are determined according to the colors of the overlaid portion of the medical image 6 so that the contrast therebetween is increased. As an example, tagging unit 7 may determine a color of the overlaid portion of the medical image 6. Based on the determined color, tagging unit 7 may select a color for the visual tag 8 that is different from the color of the overlaid portion. The color for the visual tag 8 is selected such that a contrast metric between the color for the visual tag 8 and color of the overlaid portion of the medical image 6 is above a preset minimum contrast threshold. This permits the physician (e.g., radiologist) to readily distinguish the visual tag 8 from the overlaid portion of the medical image 6.

The visual tag 8 comprises information relating to a predefined functionality, e.g., service, that can be applied to the medical image 6. Therefore, a predefined functionality is associated with the applied visual tag 8. In one embodiment, information relating to the applied visual tag 8 (for example, its position or type) and/or to its associated functionality is added to the metadata of the medical image 6 (in the DICOM header). So as to intuitively reflect the associated functionality, the visual tag 8 preferably includes a visual representation (e.g., logo, pattern and/or text) referring to the associated functionality. The predefined functionality aims at enriching functionalities provided by the medical image viewer 5 and/or overcoming one of its weaknesses. The associated functionality may be modality-specific. In some instances, the tagging unit 7 may determine which functions or services should be suggested through tags to be superimposed on medical images. The determination may be based on one or more sets of rules to create correspondence (e.g., links) between image characteristics and suggested services/functionalities. The image characteristics may be based on analysis of image data of the medical images using deep learning algorithms. Additionally or alternatively, the rules may also be based on corresponding metadata (e.g., DICOM tags). In one illustrative example, a medical image may illustrate a non-contrast head CT. The set of rules may be used to determine that a relevant functionality for the image may be calculation of a volume of an intracranial hemorrhage. A suggested tag may encode identifiers of such function and the image or series of images. The reading of the tag will trigger the calculation of the volume of the intracranial hemorrhage.

An application unit 9 is able to identify the applied visual tag 8 when the tagged medical image 6 is displayed by the medical image viewer 5. The application unit 9 is further able to apply the functionality associated with the identified visual tag based on information contained therein. The visual tag 8 includes information allowing the application unit 9 to execute or run the functionality associated thereto. Such information may include, in raw or encoded form, an Uniform Resource Identifier, an Uniform Source Locator, a query, an API request, a query/retrieve request, a command, a predefined function call, an identifier referring to a predefined function or computer program product, input data for applying the functionality, or any combination thereof.

The identification of the visual tag 8 may be based on its form, its colors, its content, its location on the medical image 6, and/or any other predefined distinguishing graphical feature.

In one embodiment, the application unit 9 comprises mobile user equipment 10. The mobile user equipment 10 includes an image sensor (namely, a camera) for capturing the visual tag 8 or, more generally, an image of at least a portion of the displayed tagged medical image comprising the visual tag. The mobile user equipment 10 further comprises a mobile application for identifying the visual tag 8 contained in the captured image, and for determining a feature from the identified visual tag information relating to a functionality associated therewith. Accordingly, when a visual tag 8 displayed by the medical image viewer 5 is scanned by the image sensor of the mobile user equipment 10, the mobile application may identify it and determine its associated functionality. Once determined, this functionality may be applied by the mobile application. The mobile user equipment 10 can be a smartphone, a tablet PC or any other mobile device provided with an image sensor. The mobile application may apply the functionality by invoking a service to be executed on a particular server or other electronic device. For instance, the mobile application may send, to the server or electronic device, identifiers of functions associated with the tag and the identifier of the medical image. The server or electronic device may then retrieve the medical image from the medical image database system 3 using the medical image identifier. The server or electronic device may then apply the functions to the retrieved medical images using programs/instructions associated with the functions.

In one embodiment, the mobile application triggers automatically the determined functionality based on information contained in the scanned and identified visual tag 8. In another embodiment, the mobile application requests user confirmation to trigger the determined functionality.

In one embodiment, the visual tag 8 comprises a query/retrieve request. The query may include identifiers of functions and, optionally, the identifier of the medical image and/or patient. The query/retrieve request is to be addressed to an application server. The query/retrieve request is, in one embodiment, to be addressed to the tagging unit 7, the medical image database system 3, or processing units 11 supporting the tagging unit 7 or a networked computing device accessed via the Internet. The request may relate to a remote execution of the functionality and/or data retrieving. The tagging unit 7 and/or the medical image database system 3 are set up to enable such remote communication from the mobile user equipment 10. The remote access may be supported by VPN connections. In another embodiment, the tagging unit 7 includes an access control module to assure users' authorization before providing a response to a received request from the mobile user equipment 10.

In one embodiment, the mobile application comprises a web-based medical image viewer providing, compared to the medical image viewer 5, advanced functionalities for medical image visualization and/or annotation (such as, multi-window layout option to display the currently displayed medical image 6 by the medical image viewer 5 with a plurality of others medical images associated therewith and retrieved from the medical image database system 3, or 3D reconstruction). Therefore, based on the information contained in the identified visual tag 8, the mobile application queries and retrieves from the medical imaging network 1 the required medical files to which the functionality associated with the identified visual tag 8 is applied. The tagging unit 7 provides an interface (or a bridge) between the mobile user equipment 10 (using a web protocol) and the medical image database system 3 (using a PACS/DICOM protocol). Accordingly, the tagging unit 7 expands the data flow to incorporate advanced web-based viewer within a DICOM network.

The functionality associated with the identified visual tag 8 can be, for instance,
an export of the displayed tagged medical image 6 to a second medical image viewer (e.g., the mobile user equipment or another device) different from the medical image viewer 5 of the user terminal 4;
a classification (or a structure recognition or delimitation) of the displayed tagged medical image 6 or of a marked region of interest of the medical image 6 according to a predefined classification algorithm (such as a machine learning classification algorithm) performed the by processing units 11 (e.g., use a deep learning algorithm to characterize a displayed growth on a patient as benign or malignant);
an area or volume calculation of a region of interest marked on the displayed tagged medical image 6 to be performed by the mobile application or by the processing units 11;
an application by the processing units 11 of a predefined viewing functionality or image processing (color inversion, image filtering such as threshold, Sharpen or Sobel filters) on a copy of the currently displayed tagged medical image 6 and uploading/storing the copy in the medical image database system 3. The copy may be later retrieved and displayed by the medical image viewer 5 to assist physicians in diagnosis (for example, highlight a specific structure). In some cases, the tag may encode a concatenated identifier of one service and an identifier of another service. In some cases, a tag may encode and trigger the display of a menu by the mobile device. The user may then select which functionalities to execute. To this end, the mobile application may store the functionalities listed in the menu. Alternatively, the mobile application may store a correspondence table defining which services are to be executed according to the information encoded by the tag. In some instances, if there are several tags attached to an image, each tag may encode all the relevant information relating to a specific functionality. The mobile application does not itself have to store the functionalities;
an insertion of information relating to the displayed tagged medical image 6 (such as a report or information retrieved from the medical image metadata) into a new email message to be sent or into a database supported by the mobile application. For example, such inserted information may include presence or absence of a pathology, post-treatment additional information, pre-diagnostic information, urgency information, etc.

Processing units 11 are application servers including programs for processing the medical images 6. Processing units 11 operate on a copy of the medical image 6. Medical images 6 generated by processing units 11 are transmitted to the medical image database system 3. A medical image 6 generated by processing units 11 may be appended to the original one as a new series within the same study to avoid disrupting existing workflows. In one embodiment, information relating to the applied processing on a copy of a medical image 6 is appended to its metadata.

The above-described embodiments to enable application of a functionality to the medical image 6 are independent from the platform supporting the medical image viewer 5. There is no requirement for modifying the medical image viewer 5, or for installing additional software on the platform supporting it.

Advantageously, these embodiments enable ubiquitous access to medical image database system 3 and increase the number of functionalities that can be applied to the medical image 6 so that radiology workflow and diagnostic efficiency are improved.

As a variant or in combination, the application unit 9 comprises a software application installed on or contained in the user terminal 4. In other words, the medical image viewer 5 and at least a component of the application unit 9 are both contained in the user terminal 4. The software application is configured to detect and identify the visual tag 8 when the tagged medical image 6 is displayed by the medical image viewer 5. In this case, the software application may be a standalone software application or, when the medical image viewer 5 is supported by a web browser (web-based), a plug-in or an add-on associated with the web browser. The software application is configured to detect the presence of the visual tag 8 in a displayed medical image 6 by the medical image viewer 5. Such detection may be achieved by analyzing (or parsing) metadata of the displayed medical image 6 indicating the presence of the visual tag 8 and/or based on at least one screenshot of the graphical user interface of medical image viewer 5.

When it is determined that the displayed medical image 6 includes an identified visual tag 8, the software is further configured to detect (observe or monitor) a predefined user interaction with the identified visual tag 8. The predefined user interaction can be, for example, a right (or left) mouse-click, a double clicking of the right (or left) mouse button or, when the user equipment has touch support, a simple or multi-touch gesture. The detection and identification of the visual tag 8 and user interaction observation by the software application may be executed as background tasks on the user terminal 4.

When a predefined user interaction with the identified visual tag 8 is detected, the application unit 9 applies, to the currently displayed tagged medical image 6, the functionality associated with the visual tag 8 based on information included in the visual tag 8. For instance, the associated functionality is, as described above, a request to be addressed to the tagging unit 7 or to the medical image database system 3 or a functionality to be executed locally on the user terminal 4 such as exporting DICOM files to local disk in PDF format with structured report/annotations, encapsulation and report in an email the DICOM files, burning displayed medical image 6 to patient-CD, compression or conversion of the displayed medical image 6 to multiple image or video formats (for instance, Jpeg, Tiff, Png, WMV, AVI), and the like.

In one embodiment, a plurality of visual tags 8 is applied to an incoming medical image 6. A predefined functionality is associated with each visual tag 8 as described above. In another embodiment, a plurality of functionalities are associated with a single visual tag 8. In the latter case, the application unit 9 is configured to apply a functionality chosen from the plurality of functionalities according to the use case scenario, or ask the user of the user terminal 4 or mobile user equipment 10 to select a functionality from the plurality of functionalities listed in a menu. A use case scenario may be defined by the acquisition modality of the medical image 6, the user session (radiologist, physician and operator), metadata of the medical image 6 (for instance patient information, imaged body part, previous report), or settings in the application unit 9.

In one embodiment, the medical imaging equipment 2 comprises the tagging unit 7 so that generated medical images 6 are directly tagged by a visual tag 8.

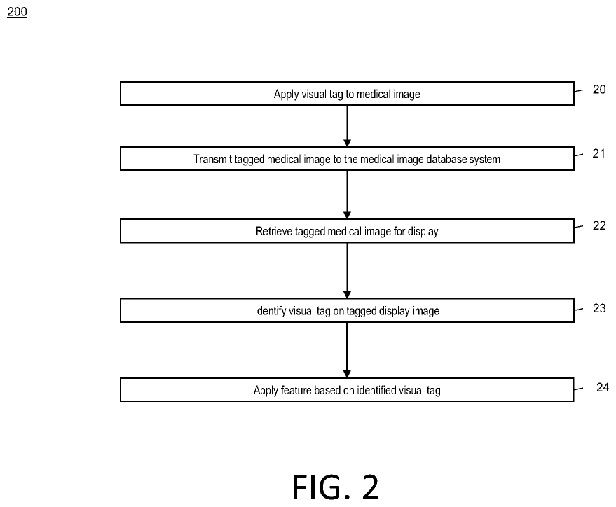

With reference to FIG. 2, there are shown process steps of a method 200 to enable application of a functionality to the medical image 6 when displayed by the medical image viewer 5. To that end, a visual tag 8 is firstly applied by the tagging unit 7 to the medical image 6 (step 20). This tagging step may take place as soon as the medical image 6 is generated by the medical imaging equipment 2, or later on request. As described above, the visual tag 8 is applied to the medical image 6 so that the visual tag 8 is visible when the tagged medical image 6 is displayed by the medical image viewer 5. The visual tag 8 contains information which enables application of the desired functionality to the medical image 6.

The tagged medical image 6 is then transmitted to the medical image database system 3 (step 21). When a physician retrieves (step 22) the tagged medical image 6 from the medical image database system via the medical image viewer 5, the visual tag 8 is therefore visible to him/her. To apply the functionality to the displayed tagged medical image 6, the physician uses the application unit 9 for the identification (step 23) of the visual tag 8. Based on information contained in the identified visual tag 8, the functionality may be applied (step 24) to the medical image 6. As illustrated above, the physician can use a smartphone including a camera and a dedicated mobile application to, respectively, scan the visual tag 8 and execute the associated functionality. In another embodiment, the physician may use a software application installed on the same user terminal 4 as the medical image viewer 5. The software application is configured to detect and identify the visual tag 8 when the tagged medical image is displayed by the medical image viewer 5, and to detect a predefined user interaction with the identified visual tag 8. As soon as the predefined user interaction with the identified visual tag 8 is detected, the functionality associated therewith can be executed based on information contained in the visual tag 8.

As used herein, the term user, radiologist or physician is meant broadly and not restrictively, to include any person who utilizes a medical image viewer, especially a proprietary medical image viewer, to retrieve and visualize a medical image.

Advantageously, the above-described embodiments facilitate the enrichment of medical image viewers with additional functionalities that meet customized needs of physicians during medical image review and reporting. Providing medical image viewers with relevant functionalities to use-case scenarios improves the revision workflow, increases physician productivity and report relevance, and optimizes diagnostic work duration specifically in time-sensitive cases where quick diagnosis is a key factor.

Advantageously, the above-described embodiments provide DICOM viewers with a broad access to convenient functionalities while preserving physician's workspace/environment (usual graphical user interfaces) without impairing user experience. Usual graphical user interfaces with which a physician/radiologist is familiar are maintained. Radiologists can improve their workflow by taking advantage of advanced functionalities and ubiquitous applications, while using their usual graphical interfaces. Additionally, the functions and services associated with the tags may be implemented without having to update the user terminal 4.

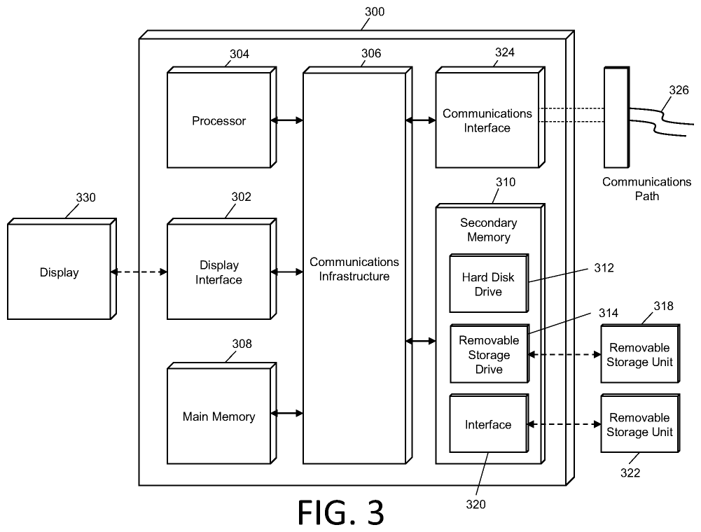

FIG. 3 illustrates a computer system 300 in which embodiments of the present disclosure, or portions thereof, may be implemented as computer-readable code. For example, one or more (e.g., each) of the medical imaging equipment 2, tagging unit 7, processing units 11, medical image database system 3, user terminal 4, mobile user equipment 10, and other device described herein may be implemented in the computer system 300 using hardware, software, firmware, non-transitory computer readable media having instructions stored thereon, or a combination thereof and may be implemented in one or more computer systems or other processing systems. Hardware, software, or any combination thereof may embody modules and components used to implement the method of FIG. 2.

If programmable logic is used, such logic may execute on a commercially available processing platform configured by executable software code to become a specific purpose computer or a special purpose device (e.g., programmable logic array, application-specific integrated circuit, etc.). A person having ordinary skill in the art may appreciate that embodiments of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device. For instance, at least one processor device and a memory may be used to implement the above-described embodiments.

A processor unit or device as discussed herein may be a single processor, a plurality of processors, or combinations thereof. Processor devices may have one or more processor “cores.” The terms “computer program medium,” “non-transitory computer readable medium,” and “computer usable medium” as discussed herein are used to generally refer to tangible media such as a removable storage unit 318, a removable storage unit 322, and a hard disk installed in hard disk drive 312.

Various embodiments of the present disclosure are described in terms of this example computer system 300. After reading this description, it will become apparent to a person skilled in the relevant art how to implement the present disclosure using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 304 may be a special purpose or a general purpose processor device specifically configured to perform the functions discussed herein. The processor device 304 may be connected to a communications infrastructure 306, such as a bus, message queue, network, multi-core message-passing scheme, etc. The network may be any network suitable for performing the functions as disclosed herein and may include a local area network (LAN), a wide area network (WAN), a wireless network (e.g., Wi-Fi), a mobile communication network, a satellite network, the Internet, fiber optic, coaxial cable, infrared, radio frequency (RF), or any combination thereof. Other suitable network types and configurations will be apparent to persons having skill in the relevant art. The computer system 300 may also include a main memory 308 (e.g., random access memory, read-only memory, etc.), and may also include a secondary memory 310. The secondary memory 310 may include the hard disk drive 312 and a removable storage drive 314, such as a floppy disk drive, a magnetic tape drive, an optical disk drive, a flash memory, etc.

The removable storage drive 314 may read from and/or write to the removable storage unit 318 in a well-known manner. The removable storage unit 318 may include a removable storage media that may be read by and written to by the removable storage drive 314. For example, if the removable storage drive 314 is a floppy disk drive or universal serial bus port, the removable storage unit 318 may be a floppy disk or portable flash drive, respectively. In one embodiment, the removable storage unit 318 may be non-transitory computer readable recording media.

In some embodiments, the secondary memory 310 may include alternative means for allowing computer programs or other instructions to be loaded into the computer system 300, for example, the removable storage unit 322 and an interface 320. Examples of such means may include a program cartridge and cartridge interface (e.g., as found in video game systems), a removable memory chip (e.g., EEPROM, PROM, etc.) and associated socket, and other removable storage units 322 and interfaces 320 as will be apparent to persons having skill in the relevant art.

Data stored in the computer system 300 (e.g., in the main memory 408 and/or the secondary memory 310) may be stored on any type of suitable computer readable media, such as optical storage (e.g., a compact disc, digital versatile disc, Blu-ray disc, etc.) or magnetic tape storage (e.g., a hard disk drive). The data may be configured in any type of suitable database configuration, such as a relational database, a structured query language (SQL) database, a distributed database, an object database, etc. Suitable configurations and storage types will be apparent to persons having skill in the relevant art.

The computer system 300 may also include a communications interface 324. The communications interface 324 may be configured to allow software and data to be transferred between the computer system 300 and external devices. Exemplary communications interfaces 324 may include a modem, a network interface (e.g., an Ethernet card), a communications port, a PCMCIA slot and card, etc. Software and data transferred via the communications interface 324 may be in the form of signals, which may be electronic, electromagnetic, optical, or other signals as will be apparent to persons having skill in the relevant art. The signals may travel via a communications path 326, which may be configured to carry the signals and may be implemented using wire, cable, fiber optics, a phone line, a cellular phone link, a radio frequency link, etc.

The computer system 300 may further include a display interface 402. The display interface 302 may be configured to allow data to be transferred between the computer system 300 and external display 330. Exemplary display interfaces 302 may include high-definition multimedia interface (HDMI), digital visual interface (DVI), video graphics array (VGA), etc. The display 330 may be any suitable type of display for displaying data transmitted via the display interface 302 of the computer system 300, including a cathode ray tube (CRT) display, liquid crystal display (LCD), light-emitting diode (LED) display, capacitive touch display, thin-film transistor (TFT) display, etc.

Computer program medium and computer usable medium may refer to memories, such as the main memory 308 and secondary memory 310, which may be memory semiconductors (e.g., DRAMs, etc.). These computer program products may be means for providing software to the computer system 300. Computer programs (e.g., computer control logic) may be stored in the main memory 308 and/or the secondary memory 310. Computer programs may also be received via the communications interface 324. Such computer programs, when executed, may enable computer system 300 to implement the present methods as discussed herein. In particular, the computer programs, when executed, may enable processor device 304 to implement the methods illustrated by FIG. 2, as discussed herein. Accordingly, such computer programs may represent controllers of the computer system 300. Where the present disclosure is implemented using software, the software may be stored in a computer program product and loaded into the computer system 300 using the removable storage drive 314, interface 320, and hard disk drive 312, or communications interface 324.

The processor device 304 may comprise one or more modules or engines configured to perform the functions of the computer system 300. Each of the modules or engines may be implemented using hardware and, in some instances, may also utilize software, such as corresponding to program code and/or programs stored in the main memory 308 or secondary memory 310. In such instances, program code may be compiled by the processor device 304 (e.g., by a compiling module or engine) prior to execution by the hardware of the computer system 300. For example, the program code may be source code written in a programming language that is translated into a lower level language, such as assembly language or machine code, for execution by the processor device 304 and/or any additional hardware components of the computer system 300. The process of compiling may include the use of lexical analysis, preprocessing, parsing, semantic analysis, syntax-directed translation, code generation, code optimization, and any other techniques that may be suitable for translation of program code into a lower level language suitable for controlling the computer system 300 to perform the functions disclosed herein. It will be apparent to persons having skill in the relevant art that such processes result in the computer system 300 being a specially configured computer system 300 uniquely programmed to perform the functions discussed above.

1. A system to enable application of a function or service to a medical image when displayed by a medical image viewer, said system including:
a tagging unit configured to apply a visual tag on said medical image so that the visual tag is visible when the medical image is displayed by the medical image viewer, said visual tag comprising information relating to said function or service; and
an application unit configured to identify the applied visual tag when the tagged medical image is displayed by the medical image viewer, said application unit being further configured to apply said function or service to the medical image based on information contained in the identified visual tag.

2. The system of claim 1, wherein the application unit comprises a mobile user equipment including:
an image sensor for capturing an image of at least a portion of the displayed tagged medical image, said at least a portion comprising the visual tag; and
a mobile application for identifying the visual tag contained in the captured image, and for determining from the identified visual tag information relating to said function or service, the mobile application being configured to apply said function or service.

3. The system of claim 1, further comprising a user terminal, the user terminal comprising the medical image viewer, the user terminal further comprising the application unit.

4. The system of claim 3, wherein the application unit comprises a software application configured to detect and identify the visual tag when the tagged medical image is displayed by the medical image viewer, said software application being further configured to detect a predefined user interaction with the identified visual tag, said software application being configured to apply said function or service when the predefined user interaction is detected.

5. The system of claim 4, wherein the medical image viewer is supported by a web browser, the software application being a plug-in associated with the web browser.

6. The system of claim 1, wherein the medical image is a Digital Imaging and Communications in Medicine object.

7. The system of claim 1, wherein the information relating to the function or service comprises a query/retrieve request.

8. The system of claim 1, wherein the medical image comprises a top layer, the visual tag being applied to the top layer.

9. A method to enable application of a function or service to a medical image when displayed by a medical image viewer, said method including the following steps:
applying a visual tag to said medical image so that the visual tag is visible when the medical image is displayed by the medical image viewer, said visual tag comprising information relating to said function or service;
identifying the applied visual tag when the tagged medical image is displayed by the medical image viewer; and
applying said function or service to the medical image based on information contained in the identified visual tag.

10. The method of claim 9, further comprising the following steps:
capturing an image of at least a portion of the displayed tagged medical image, said at least a portion comprising the visual tag;
identifying the visual tag contained in the captured image; and
determining, from the identified visual tag information relating to said function or service, said function or service.

11. The method of claim 9, further comprising the following steps:
detecting and identifying the visual tag when the tagged medical image is displayed by the medical image viewer; and
detecting a predefined user interaction with the identified visual tag, said function or service being applied to the medical image when the predefined user interaction is detected.

12. A medical imaging network comprising the system of claim 1, a medical imaging equipment, a medical image database system, and a medical image viewer.

13. The medical imaging network of claim 12, wherein said network is a Digital Imaging and Communications in Medicine network.
